(19) United States
(12) Patent Application Publication
(10) Pub. No.: US 2004/0053220 A1
(43) Pub. Date: Mar. 18, 2004

(54) Replication assay for detecting anti-viral substances

(76) Inventors: Arnold Paessens, Haan (DE); Frank Dittmer, Wuppertal (DE)

Correspondence Address: CONNOLLY BOVE LODGE & HUTZ, LLP, P O BOX 2207, WILMINGTON, DE, 19899, US

(21) Appl. No.: 10/450,793

(22) PCT Filed: Dec. 11, 2001
(86) PCT No.: PCT/EP01/14516

(30) Foreign Application Priority Data
Dec. 18, 2000 (DE) 100 63 111.8

(51) Int. Cl. C12Q 1/70

(52) U.S. Cl. 435/5

(57) ABSTRACT

The present invention relates to a replication assay for detecting antivirally effective inhibitors as a single-tube assay, comprising the combination of a natural virus isolate, suspended target cells and cell culture medium containing foetal calf serum, in which the foetal calf serum concentration is less than 5% by volume.

The present invention relates to a replication assay for finding antivirally effective inhibitors, comprising the combination of a natural virus isolate, suspended target cells and cell culture medium containing foetal calf serum, in which the foetal calf serum concentration is less than 5% by volume.

The search for novel, more active and better tolerated active substances which can be used as therapeutics for treatment in viral infections, in particular infection with HIV, is an urgent task for pharmaceutical research, which involves the need for hundreds of thousands of test compounds being studied for their effectiveness.

For this purpose, the action on virus-specific transcriptases or proteases is usually studied in an enzymatic assay, or the action of the test substance on the native unaltered virus and its behaviour is studied on suitable target cells in an in vitro model.

The large number of samples to be tested causes considerable logistical problems which can be solved only by automating the test procedures.

This is state of the art in the case of the enzymatic assays, where a large amount of samples can be studied using “high throughput screening methods” (HTS methods). Assays of this kind have also been carried out in the case of HIV enzymes. Thus, an assay on the viral enzyme integrase has been carried out recently by means of HTS (Yves Pommier et al. Antiviral Research 47 (2000) 139-148; Hazuda et al. J. Virol. 71, 7005-7011; Hazuda et al. Antiviral Chem. Chemother. 10, 63-70).

Assays on the basis of proteins and enzymes are particularly well suited to be carried out in HTS, because for the available readouts accurate and rapid evaluation possibilities are present, for example as optical methods through fluorescence measurement. A good signal-to-noise ratio can be achieved by selecting suitable colour indicators. However, the enzymatic assay in principle has the disadvantage that it includes only a small part of all viral and cellular systems involved in the replication cycle.

In contrast, the study of virus replication in cell culture allows simultaneous checking of all steps involved in virus propagation under essentially natural conditions (replication assay).

Therefore, replication assays are particularly suited to detect also unknown, virus-encoded and/or host-related processes which are in principle non-detectable in an enzymatic assay.

With respect to HIV, the said processes are, for example, attachment to and penetration into the cell to be infected and the action of virus-specific regulatory mechanisms or processes in which viral and cellular factors interact.

Such viral steps which proceed in vitro, if at all, only with the addition of extraordinarily complex reaction additives are reproduced in suitable host cells in the replication assay and function optimally within the HIV replication process.

Moreover, the replication assay provides at the same time information on the cytotoxicity (selectivity index) of the test compounds and thus a first measure for estimating the potential of the active compound.

Against this background, the replication assay is particularly suited to find novel more effective and better tolerated HIV inhibitors.

However, the replication assay has the principal disadvantage that the endpoint, that is evaluation of the behaviour of the cell exposed to the virus, cannot be determined in the HTS method as yet. Although it is common to use genetically modified HIV variants which have been provided with suitable indicator systems such as, for example, the luciferase gene, such HIV variants which have been modified by inserting reporter genes do not necessarily behave like unmodified HIV strains.

Present replication assays which are used in vitro based on the propagation of unmodified HIV are time- and resource-consuming and cannot be automated as would be required for studying a large number of test substances, and are thus unsuitable in the form presently known for assaying large substance libraries.

The reason for this is above all the in vitro behaviour of vital HIV. HIV induces on various target cells, preferably on cells of the immune system, such as H9 T lymphocytes, for example, a characteristic cytopathic effect which is depicted in the light microscope as syncytium formation, that is the virus-induced formation of large cell aggregates. In present methods, this syncytium formation is the usually customary endpoint for evaluating the cytopathic effect of HIV in cell cultures and must be observed in the microscope after a particular period of time. This way of determining virus propagation or inhibition thereof is subjective and very time-consuming. The light-microscopic analysis of the endpoint is therefore unsuitable for bulk testing on the industrial scale.

In addition to a direct microscopic analysis, further known methods for evaluating HIV-specific modifications in cell cultures are “secondary assays” such as measuring virus-specific proteins (e.g. in ELISA methods) or enzyme activities (e.g. HIV reverse transcriptase) or viral nucleic acid (e.g. PCR) from cell culture supernatants.

All these methods, however, are extraordinarily resource-consuming and therefore unsuitable for assaying large substance libraries.

It is therefore the object of the invention to establish a replication assay suitable for high throughput screening (HTS) which, using natural HIV and suspended host cells such as, for example, human T lymphocytes kept in permanent cell lines, can be carried out as primary assays using an optical readout with good signal-to-noise ratio.

According to the invention, suitable replication assays mean those assays which can be carried out as single-tube assays without further work-up steps using suspension cells.

Within the scope of the invention it has now surprisingly been found that HIV and in particular the LAV isolate of HIV propagate, even in the presence of substantially lower serum concentrations than those used hitherto in the prior art. Hitherto common growth conditions for HIV host cells were culture media which contained at least 20% foetal calf serum (FCS).

This is particularly important, since high serum concentrations in cell culture media of viral replication systems on suspension cells are unsuitable for optical detection methods based on fluorescence or absorption measurement methods, the reason of which is the presence of interfering proteins in FCS and the enzymatic activity coming from them. The application of optical detection methods therefore has been restricted hitherto to cell culture systems with adhering cells from which the proteins interfering with the optical assay, which are in the culture supernatant, can readily be removed by pipetting.

The present studies have shown now that also markedly lower serum concentrations are sufficient in order to propagate HIV and in particular the LAV isolate of HIV. In fact, it has now surprisingly been found that HIV propagates in suspension cells also at hitherto unestablished low FCS serum concentrations such as, for example, 0.2 to 2%, a concentration at which the interfering proteins contained in FCS no longer interfere with some of the optical detection methods.

Based on this observation, the present invention presents an (HTS) screening method for the rapid, cost-effective and infection-relevant testing of large substance libraries with more than 200,000 test compounds, preferably with more than 500,000 test compounds or even larger substance libraries with more than 1 million test compounds, with the aim of finding novel HIV inhibitors, which method is based on the fact that the replication assay for in vitro HIV propagation which is known from the literature and which has hitherto been usable only on a small scale also for testing active compounds against HIV can now also be carried out as HTS, after specific modifications of the assay conditions.

Within the scope of the invention it has further been found that there are HIV isolates which under the low serum concentrations of the invention disturb and influence the host cells after infection such that metabolic processes are altered, and which can then be used for detecting virus-destroying processes.

An example of an HIV isolate which can be used in connection with low FCS concentrations according to the invention is the LAV isolate of HIV, which alters the H9 T-lymphocyte cell lines such that virus infections can be visualized via altered biochemical processes in the optical assay by means of colour reagents, since infection of H9 cells with the LAV isolate of HIV in the presence of low FCS concentrations leads to the infected cells being transformed into a physiological state which makes it possible to discriminate between protected cells and cells destroyed by the virus with the use of colour reagents.

Surprisingly, colour reagents suitable for detection under these conditions were found to be XTT, MTT, neutral red and in particular alamarBlue (alamarBlue™, Laboserv GmbH, Giessen, Germany; U.S. Pat. No. 5,501,959) or fluorescent dyes such as fluorescein diacetate, for example.

A precondition for this type of detection is the surprise finding that some infection-relevant HIV strains such as, for example, the LAV isolate also propagate in cells whose culture medium contains only small amounts of foetal calf serum (FCS) as essential growth factor. This finding is an essential precondition for the method of the invention, since the relatively high FCS concentrations hitherto deemed essential interfere with and therefore exclude the use of colour reagents.

Detection of the cytopathic and cytotoxic effects induced by the LAV isolate with the FCS concentrations of the invention in connection with the colour reagents of the invention makes it now possible to carry out an HTS for detecting active substances against HIV as replication assay.

Furthermore, it was surprisingly found that some HIV isolates, in particular the LAV isolate, for example, specifically damaged the host cells in vitro so that subsequent detection of damage becomes possible with the aid of dyes which otherwise cannot be used in the methods described.

It has further been found that the dyes which can be used according to the invention are alamarBlue and fluorescence dyes such as, for example, fluorescein diacetate, which dyes can be measured on suspension cells with the aid of optical methods using the HTS technique.

It has further been found that the inventive combination comprising natural HIV isolate, cell culture medium with low FCS concentration according to the invention, detection dye of the invention and suspended H9 T lymphocytes can be configured in an HTS as single-step method such that the said method can be carried out in 384-well or else 1536-well plates.

It has further been found that the inventive combination comprising natural HIV isolate, cell culture medium with low FCS concentration according to the invention, detection dye of the invention and suspended H9 T lymphocytes can be configured in an HTS as single-step method such that the said method can be used for rapid and cost-effective detection of novel active substances against HIV.

Novel active substances in accordance with the invention are preferably low molecular weight active substances which intervene in the HI virus replication and specifically inhibit its propagation, low molecular weight meaning within the scope of the invention compounds having a molecular weight in the range from about 100 to 500 or 100 to 1000 and above.

The suitable potential inhibitors, however, may also be high molecular weight compounds such as, for example, antibodies which can be produced in a large number in libraries.

Furthermore, the assay is suited to checking large amounts of test samples which are obtained from patients during the daily clinical routine, such as serum samples or plasma samples of patients, whose virus titre has to be determined in order to determine the virus load for progress control and to determine when a therapy is required. Likewise, a multiplicity of test samples which come from patients who have to be checked for resistance development are obtained during the clinical routine.

However, the assay method of the invention may preferably be used for assaying large substance libraries and thus for detecting novel substance classes with anti-HIV action.

The assay methods of the invention mean in particular those assay methods in which target cells, in particular lymphocytes such as H9 T lymphocytes, which are sensitive to native viruses, in particular HI viruses, are cultured in suspension in a cell culture medium, for example RPMI medium which contains only small amounts of a growth factor, for example foetal calf serum, which is present at a concentration of from 0.1 to 5% by volume, preferably 1 to 2% by volume, in multiwell plates together with the said viruses and a test or reference substance at a temperature in the range from 20 to 40° C., preferably 30 to 40° C. and very preferably 35 to 38° C. and at a CO₂ concentration of from 2 to 8%, preferably 5%, for a period of 2-20 days, preferably 5 to 10 days, at the saturating humidity resulting for the particular temperature, subsequently a colouring agent, preferably alamarBlue or fluorescein diacetate is added as readout, and the mixture is then again incubated and subsequently optically measured in the HTS method.

The detailed procedure by way of example is such that in each case 3×10³ HIV-infected H9 cells are seeded into each well of a 384-well microtitre plate, which wells contain suitable dilutions of the test substances or reference substance.

A suitable dilution in accordance with the invention means using test substance concentrations between 0.1 and 50 μm, and preference is given to using test substance concentrations of from 1 to 20 μm.

The test plates are subsequently incubated in the incubator at 37° C. with 5% CO₂ and saturating humidity for 7 days. The antiviral effects or cellular compatibility of the test substances is then determined by adding as detection dye alamarBlue (Biosource International) or fluorescein diacetate (200 μg/ml in PBS) in an amount of 1/10 of the well content volume. This is followed, in the case of alamarBlue, by incubation under the abovementioned conditions for a further 5 to 7 hours and up to 24 hours. This is then followed by photometric evaluation by measuring absorption or emission. In the case of fluorescein diacetate, fluorescence emission is already measured after incubation for 20 to 60 min.

Under these assay conditions, an optical density of 1.9 extinction units was measured for the control cells, while the control viruses had an optical density of 0.02 extinction units. This astoundingly favourable signal-to-noise ratio of about 1:100 is an optimal precondition for carrying out an HTS assay with HIV in accordance with the object set.

In contrast, when using the high FCS concentrations hitherto customary in this technology in the HIV-infected cell culture (20% by volume FCS), extinctions of 1.5 extinction units for the control cells and 0.4 extinction units for the control viruses are found.

This low signal-to-noise ratio of about 1:4 is insufficient for carrying out a sensitive HTS.

1. Replication assay for finding antivirally effective inhibitors, comprising the combination of a natural virus isolate, suspended target cells and cell culture medium containing foetal calf serum, characterized in that the foetal calf serum concentration is less than 5% by volume.

2. Assay according to claim 1 for finding HIV inhibitors, wherein the virus isolate is an HIV isolate.

3. Assay according to claim 1 or 2 for finding HIV inhibitors, wherein the virus isolate is a natural HIV isolate.

4. Assay according to any of claims 1 to 3 for finding HIV inhibitors, wherein the virus isolate is the LAV isolate of HIV.

5. Assay according to one or more of claims 1 to 4, characterized in that the foetal calf serum concentration is less than 2% by volume.

6. Assay according to one or more of claims 1 to 5, characterized in that it contains one or more detection dyes.

7. Assay according to one or more of claims 1 to 6, characterized in that it contains alamarBlue or fluorescein diacetate as detection dyes.

8. Assay according to one or more of claims 1 to 7, characterized in that it contains H9 T lymphocytes as target cells.

9. Use of the assay according to one or more of claims 1 to 7 in HTS.

10. Use of inhibitors of viral replication, which have been found using an assay of claims 1 to 8, for producing medicaments for the prophylaxis and treatment of infections.
